(19) United States
(12) Patent Application Publication
(10) Pub. No.: US 2015/0367023 A1
(43) Pub. Date: Dec. 24, 2015

(54) A NOVEL GLASS-IONOMER CEMENT

(71) Applicant: THE UNIVERSITY OF SHEFFIELD, Sheffield (GB)

(72) Inventors: Cheryl MILLER, Sheffield (GB); Paul V. HATTON, Sheffield (GB); Felora MIRVAKILY, Sheffield (GB)

(21) Appl. No.: 14/758,602

(22) PCT Filed: Dec. 20, 2013
(86) PCT No.: PCT/GB2013/053386

§ 371 Date: Jun. 30, 2015

(30) Foreign Application Priority Data
Dec. 31, 2012 (GB) 1223509.9
Jun. 28, 2013 (GB) 1311648.8

(51) Int. Cl. A61L 24/12

(52) U.S. Cl. CPC A61L 24/12

(57) ABSTRACT

A novel, glass-ionomer cement (GIC), which is preferably bioactive, and which is both trivalent metal cation-free and magnesium-free, comprising: (i) a glass composition which is both trivalent metal cation-free and magnesium-free (and is preferably bioactive), and (ii) a polyacid.

The present invention relates to a novel glass-ionomer cement (GIC), and especially to such a GIC comprising a bioactive glass, and the uses of said cement in both human and veterinary medicine, including dentistry. In the context of the present invention, “bioactive” and like terms, when used with reference to a material, means that said material has the ability to elicit a favourable response or cause a beneficial reaction in living tissue.

Conventional GICs are formed from the combination of high molecular weight (MW) polyacids (e.g. polyacrylic acid), typically having an average MW greater than 10,000 Daltons, a basic fluoroaluminosilicate glass powder, and water. The properties of GICs result from these components and their setting reaction, surface chemistry, physical structure and bulk composition. Set GICs may be described as composites with inorganic glass particles set in a relatively insoluble calcium-aluminium hydrogel matrix.

Freshly-mixed, unset GIC is able to chemically bond directly to mineralised tissue, i.e. bone, as well as to metals, which is advantageous from both medical and dental perspectives. In simple terms, GICs set in stages, which include the following. Firstly, carboxylic acid residues on the polyacid ionise in the presence of water. Liberated protons then react with the surface of the basic glass particles to liberate cations, such as Al³⁺ and Ca²⁺, which are then able to crosslink the ionised carboxylic acid residues, thereby completing setting of the cement.

GICs have been employed extensively in restorative dentistry, e.g. in dental fillings, since the 1970s, and this long history suggests that they are amongst the most biocompatible dental materials available. Their apparent safety and history of good biocompatibility led scientists and clinicians to consider them for wider surgical application in the 1980s and 1990s as bone cements for use in, e.g. various surgical procedures in neuro-otological and skull-based surgery, such as repair of cerebrospinal fluid fistulas and skull defects. Their wider adoption has however been limited because, unfortunately, it was discovered that these cements released aluminium ions into the body that both (a) inhibited mineralization of bone, and (b) were associated with neurotoxicity. These factors severely restricted the use of these cements in medical applications to the extent that today they are used only for specific operations in otology, i.e. bone tissue repair and cementation of medical devices in ear surgery.

Few attempts have been made to generate aluminium-free glass composition for use in GICs, mainly because the technical prejudice in the field has been (and remains) to consider the presence of Al³⁺ cations as essential in order to achieve cement setting. The following summarises the state of the art prior to realization of the present invention.

In a paper entitled “Preparation of Al-Free Glass-Ionomer Cement” by M. Kamitakahara et al. published in 2000 in the Journal of the Ceramic Society of Japan, volume 108, no. 12, pages 1117-1118, prior art glass powders consisting of CaO—Al₂O₃—SiO₂—CaF₂ were modified so as to omit aluminium to form CaO—Fe₂O₃—SiO₂ glass compositions. On admixture with polyacrylic acid and water, cement formation was achieved, with acceptable working and setting times observed. It is thought that cement formation was possible due to release of Fe³⁺ and Ca²⁺ ions from the glass composition to form Fe(III) and Ca(II) polyacrylates. Thus Kamitakahara et al. essentially teaches the replacement of one trivalent cation (Al³⁺) with another trivalent cation (Fe³⁺). Subsequent studies have suggested, however, that such iron-containing glass compositions are not bioactive.

WO03/028670A1 was subsequently published in April 2003 and describes the formation of a novel polyacid cement (i.e. GIC) from an oxide powder comprising iron (III) oxide as a single oxide in the form of Fe₂O₃ or as a mixed oxide with, e.g. Fe(II)O. At least 10% by weight of Fe₂O₃ is said to be preferable. Again, the art teaches that a trivalent cation species must be present in order for a setting cement to be produced.

In December 2007, WO2007/144662A1 was published describing a bioactive glass having a number of different uses, including in a polyacid cement, the composition comprising 49-54% SiO₂, up to 1.5% P₂O₅, 7-10% CaO, 8-19% SrO, approximately 7% Na₂O, approximately 3% ZnO and 10-20% MgO (page 34, lines 4-9). The glass composition is preferably aluminium-free and free of iron oxides, e.g. Fe₂O₃ and FeO. In effect therefore, the teaching in WO '662 is to replace trivalent cations such as Al³⁺ and/or Fe³⁺ with the following specific divalent cations: Sr²⁺, Zn²⁺ and Mg²⁺. Examples 24-27 in Table 1 on page 41 are of particular note.

Finally, WO2009/004349A2 was published in January 2009 and is directed to bioactive glass compositions for use in the formation of polycarboxylate cements by an acid-base reactions of a polymer (e.g. polyacrylic acid) with an acid-leachable source of polyvalent metal ions (e.g. a fluoroaluminosilicate glass powder). Although again the glass compositions are aluminium-free and form cement compositions with non-degradable polyacids, the glass compositions comprise SiO₂ (molar % <60) and MgO (molar % >20). Thus again the teaching of the art is replacement of trivalent cations such as Al³⁺ and/or Fe³⁺ with at least Mg²⁺ as a specific divalent cation.

Such magnesium-containing glasses have been the subject of a recent review: in the paper entitled “Magnesium-Containing Bioactive Glasses for Biomedical Applications” by M. Diba et al published in 2012 in the International Journal of Applied Glass Science, volume 3, issue 3, pages 221-253, it is suggested that the presence of magnesium in bioactive glasses may actually be associated with reduced bioactivity.

It is therefore an object of the invention to provide a novel glass-ionomer cement comprising a bioactive glass composition, which does not suffer from known problems relating to bone mineralisation and neurotoxicity as have been observed in the prior art to enable wider surgical use of the cement.

Accordingly, the present invention provides a novel glass-ionomer cement, which is both trivalent metal cation-free and magnesium-free, comprising:
(i) a bioactive glass of a composition which is both trivalent metal cation-free and magnesium-free, and
(ii) a polyacid.

Said novel glass-ionomer cement preferably consists essentially of, and further preferably consists of, (i) the bioactive glass of a composition which is both trivalent metal cation-free and magnesium-free and (ii) the polyacid. Surprisingly, the present inventors discovered that such a bioactive glass composition could be formed into a GIC by reaction with a polyacid and water, despite the composition being devoid of any trivalent metal cation species (or any replacement species such as divalent MgO), the presence of which was previously thought to be critical to the setting of such cements.

Furthermore, the inventors discovered that known trivalent-metal cation free and magnesium-free bioactive glass compositions such as Bioglass™ 45 S5 glass (of composition: SiO₂: 45% wt; Na₂O: 24.5% wt; CaO: 24.5% wt and P₂O₅: 6.0% wt) also form excellent GICs on reaction with a polyacid and water.

Pleasingly, the glass-ionomer cement of the invention is believed to encourage bone healing and regeneration without exhibition of any of the adverse effects observed in the prior art on the mineralization of the new tissue.

By “trivalent metal cation-free” and “magnesium-free”, it is meant that neither of these species is present in the bioactive glass or the resultant cement in any more than a negligible amount, if present at all, with a negligible amount being less than 0.01% by weight of the glass composition. Certainly, it is not intended that either of these species be deliberately added to the bioactive glass or the resultant cement, such that any negligible amount present would be by way of inherent inclusion in the materials from which the glass is made.

The bioactive glass from which the novel, bioactive GIC is made may have a composition comprising silica (as SiO₂), sodium oxide (as Na₂O), calcium oxide (as CaO) and, optionally, phosphorus pentoxide (as P₂O₅). Preferably, the glass composition consists essentially of SiO₂, Na₂O, CaO and P₂O₅. Further preferably the glass composition consists of SiO₂ Na₂O, CaO and P₂O₅. The glass composition is preferably free from zinc or zinc oxide, meaning that, if present at all, it may only be in a negligible amount being less than 0.01% by weight of the glass composition.

The bioactive glass composition comprised in the cement may thus comprise, preferably consist essentially of, and further preferably consist of, the following amounts (in molar percentages) of components:

| | |
|---|---|
| SiO₂ | 30-63 |
| Na₂O | 5-40 |
| CaO | 10-50 |
| P₂O₅ | 0-8, |

preferably with the total amount of sodium oxide and calcium oxide being at least 30 molar percent.

More preferably, the glass composition comprises (preferably consists essentially of, further preferably consists of) the following amounts (in molar percentages) of components:

| | |
|---|---|
| SiO₂ | 42-62 |
| Na₂O | 20-29 |
| CaO | 11-28 |
| P₂O₅ | 0.5-5.5, |

preferably with the total amount of sodium oxide and calcium oxide being at least 35 molar percent.

In some embodiments, the bioactive glass comprised in a novel bioactive GIC according to the invention may further comprise strontium, particularly as SrO, so as to improve the radio-opacity of the resultant GIC having said glass composition incorporated. Strontia may be used as a complete (i.e. 100%) replacement for CaO, or in any reduced percentage as a replacement for CaO, for example in amount of up to 50 molar percent, preferably in the range of 0.5 to 25 molar percent, further preferably in the range 1 to 13.5 molar percent.

Beneficially, it seems that providing the bioactive glass in powder form having a maximum particle size of no more than 100 μm, preferably of no more than 75 μm, further preferably of no more than 50 μm, and typically of around 45 μm or less, as measured through an appropriately sized sieve, assists in achievement of a GIC with the properties hereinbefore described. Indeed, the smaller the particles size, the better the GIC is likely to be, with sub-micron particles sizes (including nanoparticles) thought to be most preferred for achieving the best performing GIC. Such particle sizes may be achieved by milling, e.g. ball-milling, the granular glass frit produced by deposition of molten glass (of the desired composition) into a cooling liquid (such as deionised water). Of course, any other suitable particle size comminution method as a person skilled in the art sees fit may be employed to achieve the desired powder particle size(s).

Preferably, the polyacid comprised in the GIC according to the invention is chosen from any one or more of the following: a homopolymer or copolymer prepared from the any of the following unsaturated carboxylic acid monomers in list (A) or a copolymer prepared from any of the unsaturated carboxylic acid monomers in list (A) and any of the unsaturated aliphatic compounds in list (B):
(A) acrylic acid, 2-chloroacrylic acid, 3-chloroacrylic acid, 2-bromoacrylic acid, 3-bromoacrylic acid, methacrylic acid, itaconic acid, maleic acid, glutaconic acid, aconitic acid, citraconic acid, mesaconic acid, fumaric acid and tiglicinic acid, and
(B) acrylamide, acrylonitrile, vinyl chloride, allyl chloride, vinyl acetate, 2-hydroxyethyl methacrylate.

Any of the homopolymers or copolymers referred to above may be branched polymers, which may further incorporate one or more polyacids other than those named in list (A) above.

A homopolymer or copolymer of acrylic acid is, however, preferable, with polyacrylic acid being most preferred, so as to achieve the desired control over both the working time and setting time of the cement, in cases where setting of the cement is to occur in situ. Where the cement is provided as pre-set granules or other such pre-set particles, of course, the working time and setting time would not be of relevance.

The “working time” is defined as the period of time, immediately post-mixing of the wet GIC mixture, in which the cement can be worked into the necessary cavity or position of interest. Preferably the working time is in the range of from 30 seconds to 6 minutes, further preferably in the range of from 1 minute to 5 minutes, and most preferably in the range of from 2 to 4 minutes.

Similarly, the “setting time” is defined as the period of time following the “working time” during which the cement is no longer feasibly workable until it has fully set. Preferably the setting time is in the range of from 30 seconds to 40 minutes, further preferably in the range of from 2 to 20 minutes, and most preferably in the range of from 3 to 10 minutes.

Advantageously, the GIC according to the invention further comprises a setting modifier, preferably in the form of a small, low molecular weight, acidic species such as phosphoric acid (H₃PO₄), itaconic acid (C₅H₆O₄) or maleic acid (C₄H₄O₄), which may extend the working time of the cement without significantly affecting the setting time of the cement, i.e. the setting time may be reduced when a setting modifier is incorporated into the GIC at or before its preparation.

Furthermore, it may be desirable to incorporate other species into the GIC formulation, including entities such as antibiotics, so as to reduce the occurrence of post-operative infection, e.g. following orthopaedic surgery, biologically active agents such as chlorhexidine, and/or biologically active molecules such as proteins. In this way, GICs according to the invention may be used as matrices/scaffolds for drug delivery in bone tissue.

Moreover, the GIC according to the invention may be comprised in a coating composition, preferably of micrometre thickness, on a substrate to provide said substrate with the benefits associated with the bioactivity of the GIC.

According to a second aspect of the invention, there is provided a two-part glass-ionomer cement kit comprising (1) a bioactive glass of a composition which is both trivalent metal cation-free and magnesium-free and (2) a polyacid in a weight ratio range of from 1:0.05 to 1:0.5 (as glass:polyacid), preferably from 1:0.08 to 1:0.3. The kit further preferably consists essentially of, and most preferably consists of (1) the bioactive glass and (2) the polyacid. One or both of (1) the bioactive glass and (2) the polyacid are preferably as hereinbefore described with reference to the first aspect of the invention.

According to a third aspect of the invention, there is provided a pre-set glass-ionomer cement comprising a bioactive glass composition which is both trivalent metal cation-free and magnesium-free and a polyacid in a weight ratio range of from 1:0.05 to 1:0.5 (as glass:polyacid), preferably from 1:0.08 to 1:0.3. The pre-set glass-ionomer cement further preferably consists essentially of, and most preferably consists of the bioactive glass composition and the polyacid. One or both of the bioactive glass composition and the polyacid are preferably as hereinbefore described with reference to the first aspect of the invention.

The pre-set GIC may be provided in the form of granules, moulded bodies (e.g. blocks, spheres or custom shapes), or any other form as is desired, containing pre-set cement particles which do not require working or setting prior to be deposited into the requisite location of use; the GIC granules, moulded bodies, etc. can be used “as is”. When in the form of granules, these may preferably have a mean particle diameter in the range of from 1 μm to 50 mm, preferably from 100 μm to 5 mm. Furthermore, said pre-set GIC may additionally be provided with a GIC-coating, of the type hereinbefore described, on some or all of its available surface area to provide enhanced and/or dual-action bioactivity.

A glass-ionomer cement, and granules, moulded bodies, coatings, etc. of the same, according to the invention have, as indicated above, a number of uses which include, but are not limited to use in, otology, in which the GIC may be used in skull base surgery (e.g. repair of bone to prevent CSF leakage), in an increased number of dental applications, such as new regenerative therapies to treat bone loss following periodontal disease and bone regeneration following other common bony defects (e.g. socket filling after extraction), and in orthopaedic applications, in which the GIC may be used as a bone graft substitute (instead of the current block, particulate or paste-like materials) or as a material for the reinforcement of osteoporotic vertebrae, or as a coating on a medical device to facilitate integration with bone tissue.

For a better understanding, the present invention will now be more particularly described in the following Examples.

The following materials were used in the Examples:
silica (>99%) was obtained from Rieden den Haan;
sodium carbonate (>99%) was obtained from Fisher Scientific;
calcium carbonate (>98%) was obtained from Acros Organics;
calcium hydrogen phosphate (>98%) was obtained from Sigma Aldrich;
strontium carbonate (>99%) was obtained from Sigma Aldrich;
poly(acrylic acid) (PAA) as a homopolymer polyacid: used in powder form; MW of 52 kDa; obtained from Advanced Healthcare Limited;
poly(acrylic acid-co-maleic acid) as a copolymer polyacid: used in solution by mixing granules (as obtained) with distilled water in a bench top mixing machine until the solution was homogeneous to obtain an equivalent concentration to the PAA;
phosphoric acid: a 50% (w/v) solution was prepared from crystalline phosphoric acid having a MW of 98.00 g/mol;
itaconic acid: used in powder form 99%) having a MW of 130.10 g/mol;
maleic acid: used in powder form 99%) having a MW of 116.07 g/mol.

### Glass Synthesis

The glass composition of Example 1 detailed in Table 1 below was prepared by mixing together 45 g of silica powder, 41.89 g of sodium carbonate, 35.27 g of calcium carbonate and 11.50 g of calcium hydrogen phosphate in a rotary mixer for 15 minutes. The mixture was then placed in a platinum crucible and melted in an electric furnace at a temperature ranging from 1400-1450° C. for 4 hours. The resulting molten glass was poured into 9 litres of deionised water to produce a granular frit, which was dried at 150° C. for 2 hours. Once dried, the glass frit was pulverized using a mortar to obtain powder particles of 2 mm approximate size, and subsequently milled for 4 hours in a planetary ball miller to further reduce particle size. The desired powder particle fraction was obtained by sieving through a 45 micron mesh sieve.

A further nine glass compositions were prepared by the same method, with varying amounts of silica, sodium oxide, calcium oxide, phosphorus pentoxide and, optionally, strontium oxide, so as to obtain the glass compositions shown in Table 1 below (with the amounts being molar percentages).

TABLE 1

| Example | SiO₂ | Na₂O | CaO | P₂O₅ | SrO |
|---|---|---|---|---|---|
| 1 | 46.14 | 24.35 | 26.91 | 2.60 | 0 |
| 2 | 43.88 | 27.34 | 26.12 | 2.65 | 0 |
| 3 | 50.94 | 22.14 | 25.60 | 1.32 | 0 |
| 4 | 50.28 | 21.85 | 25.27 | 2.60 | 0 |
| 5 | 49.00 | 21.30 | 24.62 | 5.07 | 0 |
| 6 | 52.16 | 22.25 | 22.95 | 2.65 | 0 |
| 7 | 54.65 | 22.99 | 20.62 | 1.75 | 0 |
| 8 | 60.54 | 24.28 | 12.53 | 2.65 | 0 |
| 9 | 46.13 | 24.35 | 24.22 | 2.60 | 2.69 |
| 10 | 46.13 | 24.35 | 13.46 | 2.60 | 13.46 |

### Cement Preparation

A number of the glass compositions from Table 1 were selected to be formed into glass-ionomer cements. Each cement sample was prepared by hand by mixing an amount of the chosen glass powder with an amount of a polyacid, optionally in combination with a setting modifier, in deionised water on a glass slab using a stainless steel spatula at an average room temperature of 22° C.

Once mixed, discs of 4 mm diameter and 1 mm thickness of each GIC were immediately formed in silicone moulds and allowed to set at room temperature for a period of time (the Setting Time). Once set, the discs were removed from their moulds and allowed to dry for 24 hours at 37° C., prior to their immersion in 7 ml of distilled water at a temperature of 37° C. The cement samples were monitored daily for their structural integrity, with the results being provided in Table 2 below.

TABLE 2

| | Cement Composition | | | | | Previous | Current |
|---|---|---|---|---|---|---|---|
| | | | Setting | Deionised | Setting | Stability | Stability |
| Cement | Glass Comp. | Polyacid | Modifier | Water | Time | in Water | in Water |
| Ex. | [Amount (g)] | [Amount (g)] | [Amount] | [Amount (g)] | (Min) | at 37° C. | at 37° C. |
| Cem1 | 2 | PAA | H₃PO₄ (aq) | 0.040 | — | >3 | >15 |
| | (0.125) | (0.020) | (35 μl) | | | months | months |
| Cem2 | 1 | PAA | — | 0.080 | 36.22 | >8 | >11 |
| | (0.125) | (0.010) | | | | months | months |
| Cem3 | 1 | PAA | — | 0.080 | 18.43 | >8 | >11 |
| | (0.125) | (0.015) | | | | months | months |
| Cem4 | 1 | PAA | — | 0.080 | 10.93 | >8 | >11 |
| | (0.125) | (0.020) | | | | months | months |
| Cem5 | 1 | PAA | H₃PO₄ (aq) | 0.035 | 8.72 | >8 | >20 |
| | (0.125) | (0.015) | (35 μl) | | | months | months |
| Cem6 | 1 | PAA | H₃PO₄ (aq) | 0.035 | 7.06 | >8 | >20 |
| | (0.125) | (0.020) | (40 μl) | | | months | months |
| Cem7 | 1 | PAA-co- | — | 0.043 | 8.82 | >2 | >14 |
| | (0.140) | Maleic | | | | months | months |
| | | (0.042) | | | | | |
| Cem8 | 3 | PAA | H₃PO₄ (aq) | 0.025 | — | >96 | >17 |
| | (0.125) | (0.010) | (35 μl) | | | months | months |
| Cem9 | 4 | PAA | H₃PO₄ (aq) | 0.020 | 11.99 | >6 | >18 |
| | (0.125) | (0.010) | (35 μl) | | | months | months |
| Cem10 | 4 | PAA | H₃PO₄ (aq) | 0.025 | 5.31 | >6 | >18 |
| | (0.125) | (0.020) | (35 μl) | | | months | months |
| Cem11 | 5 | PAA | H₃PO₄ (aq) | 0.020 | 12.16 | >4 | >16 |
| | (0.125) | (0.010) | (25 μl) | | | months | months |
| Cem12 | 5 | PAA | H₃PO₄ (aq) | 0.020 | — | >4 | >16 |
| | (0.125) | (0.010) | (35 μl) | | | months | months |
| Cem13 | 5 | PAA | H₃PO₄ (aq) | 0.020 | — | >4 | >16 |
| | (0.125) | (0.010) | (40 μl) | | | months | months |
| Cem14 | 4 | PAA | H₃PO₄ (aq) | 0.080 | — | >3 | >15 |
| | (0.125) | (0.020) | (50 μl) | | | months | months |
| | & 5 | | | | | | |
| | (0.125) | | | | | | |
| Cem15 | 7 | PAA | H₃PO₄ (aq) | 0.030 | 12.62 | >8 | >20 |
| | (0.125) | (0.015) | (40 μl) | | | months | months |
| Cem16 | 7 | PAA | Maleic acid | 0.070 | — | >3 | >14 |
| | (0.125) | (0.010) | (0.010 g) | | | months | months |
| Cem17 | 9 | PAA | H₃PO₄ (aq) | 0.035 | 6.56 | >2 | >14 |
| | (0.125) | (0.015) | (35 μl) | | | months | months |
| Cem 18 | 6 | PAA | H₃PO₄ (aq) | 0.035 | | — | >13 |
| | (0.150) | (0.015) | (35 μl) | | | | months |

Clearly we can see from Table 2 that all of the GICs prepared in accordance with the invention are stable (to date) in deionised water at 37° C. for at least eleven months. Indeed, the majority of the GICs formed are stable for at least fourteen months, many for at least sixteen months and a number for at least twenty months. These results clearly show that all of the GICs formed exhibit stability in water at 37° C. for almost one year, and it is envisaged that many of these, if not all, will continue to exhibit such stability for a number of years, once a suitable period of time has elapsed to allow such a longer-term determination to be made.

With such long-lasting structural stability, successful use of a GIC (either in kit or pre-set form) in otology, in an increased number of dental applications, in orthopaedic applications, and indeed in any other medical, cosmetic or veterinary procedure related to and affecting the repair or augmentation of bone tissue, is clearly envisaged, without any of the complications previously observed in the prior art.

What is claimed is:

1. A glass-ionomer cement, which is both trivalent metal cation-free and magnesium-free, comprising:
(i) a glass composition which is both trivalent metal cation-free and magnesium-free, and
(ii) a polyacid.

2. A glass-ionomer cement as claimed in claim 1 in which the glass has a composition comprising silica (as SiO₂), sodium oxide (as Na₂O), calcium oxide (as CaO) and, optionally, phosphorus pentoxide (as P₂O₅).

3. A glass-ionomer cement as claimed in claim 2 wherein the glass composition comprises the following amounts (in molar percentages) of components:

| | |
|---|---|
| SiO₂ | 30-63 |
| Na₂O | 5-40 |
| CaO | 10-50 |
| P₂O₅ | 0-8. |

4. A glass-ionomer cement as claimed in claim 2 wherein the glass composition further comprises strontium, particularly as SrO.

5. A glass-ionomer cement as claimed in claim 4 wherein strontia is present in the glass composition in an amount of up to 50 molar percent.

6. A glass-ionomer cement as claimed in claim 1, wherein the glass composition is free of zinc or zinc oxide.

7. A glass-ionomer cement as claimed in claim 1 in which the glass is provided in powder form having a maximum particle size of 100 μm or less.

8. A glass-ionomer cement as claimed in claim 1 wherein the polyacid is chosen from any one or more of the following: a homopolymer or copolymer prepared from the any of the following unsaturated carboxylic acid monomers in list (A) or a copolymer prepared from any of the unsaturated carboxylic acid monomers in list (A) and any of the unsaturated aliphatic compounds in list (B):
(A) acrylic acid, 2-chloroacrylic acid, 3-chloroacrylic acid, 2-bromoacrylic acid, 3-bromoacrylic acid, methacrylic acid, itaconic acid, maleic acid, glutaconic acid, aconitic acid, citraconic acid, mesaconic acid, fumaric acid and tiglicinic acid, and
(B) acrylamide, acrylonitrile, vinyl chloride, allyl chloride, vinyl acetate, 2-hydroxyethyl methacrylate.

9. A glass-ionomer cement as claimed in claim 1 wherein the polyacid is a homopolymer or copolymer of acrylic acid.

10. A glass-ionomer cement as claimed in claim 1 wherein the polyacid is polyacrylic acid.

11. A glass-ionomer cement as claimed in claim 1 wherein the working time of the cement is in the range of from 30 seconds to 6 minutes.

12. A glass-ionomer cement as claimed in claim 1 wherein the setting time of the cement is in the range of from 30 seconds to 40 minutes.

13. A glass-ionomer cement as claimed in claim 1 further comprising a setting modifier.

14. A glass-ionomer cement as claimed in claim 13 wherein the setting modifier is a low molecular weight acid selected from phosphoric acid (H₃PO₄), itaconic acid (C₅H₆O₄) and maleic acid (C₄H₄O₄).

15. A glass-ionomer cement as claimed in claim 1 further comprising any one or more of the following additional species: antibiotics, biologically active agents and biologically active molecules.

16. A coating composition for a substrate comprising a glass-ionomer cement according to claim 1.

17. A two-part glass-ionomer cement kit comprising:
(1) a glass of a composition which is both trivalent metal cation-free and magnesium-free; and
(2) a polyacid in a weight ratio range of from 1:0.05 to 1:0.5 (as glass:polyacid).

18. Pre-set glass-ionomer cement comprising:
a glass composition which is both trivalent metal cation-free and magnesium-free; and
a polyacid in a weight ratio range of from 1:0.05 to 1:0.5 (as glass:polyacid).

19. Pre-set glass-ionomer cement as claimed in claim 18 provided in the form of granules, moulded bodies or any other form as is desired containing pre-set cement particles.

20. Pre-set glass-ionomer cement as claimed in claim 19 wherein said granules have a mean particle diameter in the range of 1 μm to 50 mm.

21. Pre-set glass-ionomer cement as claimed in claim 18 provided with a coating composition for a substrate comprising a glass-ionomer cement which is both trivalent metal cation-free and magnesium-free, comprising:
(i) a glass composition which is both trivalent metal cation-free and magnesium-free, and
(ii) a polyacid.

22. (canceled)
